(19) United States
(12) Patent Application Publication
(10) Pub. No.: US 2019/0117864 A1
(43) Pub. Date: Apr. 25, 2019

(54) METHOD OF ATTACHING TISSUE SUBSTITUTE ARTIFICIAL FIBER CLOTH, CONNECTING CABLE FOR AUXILIARY ARTIFICIAL HEART, AND AUXILIARY ARTIFICIAL HEART SYSTEM

(71) Applicant: SUN MEDICAL TECHNOLOGY RESEARCH CORPORATION, Suwa-shi, Nagano (JP)

(72) Inventor: Masami UCHIDA, Nagano (JP)

(21) Appl. No.: 16/094,469

(22) PCT Filed: Apr. 19, 2016
(86) PCT No.: PCT/JP2016/062446

§ 371 Date: Oct. 18, 2018

(51) Int. Cl. A61M 1/12; A61M 1/10; F04D 29/12

(52) U.S. Cl. CPC A61M 1/122; F04D 29/126; A61M 1/102; A61M 1/1025

(57) ABSTRACT

A method of attaching a tissue substitute artificial fiber cloth where a fabric is attached to a cable body of a connecting cable for an auxiliary artificial heart such that the fabric covers a surface of the cable body, includes: a wrapping step of wrapping the fabric around the surface of the cable body such that the fabric extends along a longitudinal direction of the cable body, wherein long sides of the fabric have at least a length between an inlet portion of a body and a blood pump and short sides of the fabric have a length longer than a circumferential length of the cable body; an overlapping step of overlapping respective left and right marginal portions formed on long sides of the fabric; and a welding step of welding root portions of the marginal portions on a cable body side along the longitudinal direction of the cable body.

### RELATED APPLICATIONS

The present application is a National Phase of International Application Number PCT/JP2016/062446, filed Apr. 19, 2016.

### TECHNICAL FIELD

The present invention relates to a method of attaching a tissue substitute artificial fiber cloth, a connecting cable for auxiliary artificial heart, and an auxiliary artificial heart system.

### BACKGROUND ART

As medical equipment used for a patient suffering from a heavy cardiac insufficiency, there has been known an auxiliary artificial heart system which complements a part of a function of a heart (see patent literature 1, for example).

FIG. 5 is a view for describing an auxiliary artificial heart system 900 described in patent literature 1. As shown in FIG. 5, the auxiliary artificial heart system 900 described in patent literature 1 includes: a blood pump 910 embedded in a body of a patient; artificial blood vessels 920, 930 provided for connecting the blood pump 910 and a blood flow of a heart; a controller (not shown in the drawing) which has a function of controlling the blood pump 910 outside the body; and a connecting cable 950 for an auxiliary artificial heart disposed between the blood pump 910 and the controller.

The controller is housed in a portable controller accommodating part (not shown in the drawing) , for example. An electric signal line (not shown in the drawing) for controlling the blood pump 910 is disposed in the inside of the connecting cable 950. Further, in the auxiliary artificial heart system 900 described in patent literature 1, a liquid having functions such as lubrication, cooling and maintaining of a sealing performance of the inside of the blood pump 910 (also referred to as a purge liquid) is made to circulate. Accordingly, the portable controller accommodating part also includes a purge liquid circulating device for circulating the purge liquid. A purge liquid circulating tube for circulating the purge liquid (not shown in the drawing) is also disposed in the connecting cable 950.

In such an auxiliary artificial heart system 900, although it is not explicitly described in patent literature 1 particularly, with respect to the connecting cable 950, a tissue substitute artificial fiber cloth is attached to the connecting cable 950 so as to cover a surface of the connecting cable 950 at least between an inlet portion A of the body and the blood pump 910 between the blood pump 910 and the controller.

To be more specific, as shown in FIG. 6, the connecting cable 950 includes: a cable body 951 in which a control signal line for controlling the blood pump and the like are disposed; and a tissue substitute artificial fiber cloth 952 attached to the cable body 951 so as to cover a surface of the cable body 951. Since the tissue substitute artificial fiber cloth is also referred to as “fabric”, the tissue substitute artificial fiber cloth may be also expressed as fabric hereinafter. The fabric is formed using a non-woven fabric made of polyester or the like in many cases.

As shown in FIG. 6, the fabric 952 is attached to the cable body 951 such that the fabric 952 extends from the blood pump 910, passes through the inlet portion A of the body, and extends to a portion of the cable body 951 exposed outside the body. This is because a fabric used as an existing product has a width of approximately 50 mm to 60 mm and a length of approximately 300 mm in general. When the fabric having such a size is directly used, and is attached to the cable body 951 using a connecting portion 951a between the cable body 951 and the blood pump 910 as a starting point, in the case of a person having an ordinary physical build, as shown in FIG. 6, the fabric 952 is brought into a state where the fabric 952 extends from the blood pump 910, passes through the inlet portion A of the body, and is exposed outside the body. A length of a portion of the fabric 952 exposed outside the body is approximately 100 mm in general although a difference exists among individuals to some extent.

Main reasons that the fabric 952 is attached to the cable body 951 as described above are as follows. It is possible to prevent the occurrence of a case where germs or the like intrude from the inlet portion A of the body along a surface of the cable body 951 and to prevent the germs or the like from reaching the blood pump 910. It is also possible to prevent the occurrence of a case where when an external force is applied to the connecting cable 950, the external force applied to the connecting cable 950 is transmitted to the blood pump.

That is, by attaching the fabric 952 to the cable body 952, the fabric 952 and tissues such as skin and flesh around the inlet portion A of the body and tissues in the body adhere to each other thus making it difficult for germs or the like to reach the blood pump 910, and preventing the connecting cable 950 from being easily moved. Particularly, due to the adhesion of the fabric 952 and tissues such as skin and flesh at the portion around the inlet portion A of the body, the fabric 952 is integrated with the tissues such as skin and flesh so that an effect of preventing the intrusion of germs and an effect of fixing the connecting cable can be further increased.

### CITATION LIST

### Patent Literature

PTL 1: JP-A-2015-150370

### SUMMARY OF INVENTION

### Technical Problem

In attaching the fabric 952 to the cable body 951, conventionally, the fabric 952 is wrapped around the surface of the cable body 951, and portions of the fabric 952 in the vicinity of respective edge portions in a longitudinal direction are seamed to each other.

FIG. 7A and FIG. 7B are views showing a main part of the connecting cable 950 shown in FIG. 6 in an enlarged manner. FIG. 7A is a perspective view, and FIG. 7B is an enlarged cross-sectional view as viewed in an arrow direction of a-a in FIG. 7A. As described previously, in the auxiliary artificial heart system 900 described in patent literature 1, for circulating a purge liquid, in the cable body 951 of the connecting cable 950, a purge liquid circulating tube 962 is also disposed besides an electric signal line 961 for controlling the blood pump 910.

As shown in FIG. 7A and FIG. 7B, with respect to the connecting cable 950 formed by attaching the fabric 952 to the cable body 951, the respective edge portions of the fabric 952 in the longitudinal direction are respectively folded back toward the inside (a surface side of the cable body 951), and respective folded back portions 952a, 952b are seamed by a thread 960. Seaming of the folded back portions 952a, 952b is performed using a thread and a needle used in seaming in an excision operation in general.

In such a case, assuming that a length of the fabric 952 in the longitudinal direction is 300 mm, a length that the folded back portions 952a, 952b to be seamed become approximately 300 mm. Such seaming is performed in general such that a length of approximately several tens of millimeters is set as one unit (referred to as a seaming unit), and seaming is sequentially performed for respective seaming units from an end portion of the fabric 952 in a longitudinal direction. Further, before performing seaming for respective seaming units, an adhesive agent is applied by coating to the surface of the cable body 951 prior to seaming for respective seaming units.

Then, specific steps of attaching the fabric 952 to the cable body 951 are described. Firstly, an adhesive agent is applied by coating to the surface of the cable body 951 within a range corresponding to a length of 1 seaming unit amount (approximately several tens of millimeters) from the end portion of the fabric in the longitudinal direction, and seaming is performed with respect to such 1 seaming unit amount. In this case, as described previously, the respective edge portions of the fabric 952 in the longitudinal direction are folded back toward the inside, and the respective folded back portions 952a, 952b which are formed by such a folding back operation are seamed to each other in a state where the respective folded back portions 952a, 952b opposedly face each other. Such an operation is sequentially repeatedly performed for respective seaming units. The fabric 952 is thin with an average thickness of approximately 0.5 mm and hence, it is considered preferable to perform seaming by a Lembert method or the like as a seaming method.

In this manner, the above-mentioned seaming operation is sequentially repeatedly performed for respective seaming units using approximately several tens of millimeters as 1 seaming unit. However, such a seaming operation requires a skill so that in the present circumstances, even a skilled person requires 5 to 6 hours for finishing seaming over the whole length of approximately 300 mm. Further, the mechanization of such a seaming operation is difficult and hence, there is no way but to perform the seaming operation manually in the present circumstances. Accordingly, productivity is extremely small. Further, there may be also a case where even when a skilled operator performs seaming, irregularities occur in quality. Still further, the portions to be seamed are folded back and hence, as described in a frame R indicated by a broken line in FIG. 7B, the fabric 952 at the folded back portions is formed in an overlapping manner and hence, a thickness of the fabric 952 at the folded back portions becomes large locally compared to other portions.

In this manner, the conventional method of attaching a tissue substitute artificial fiber cloth (fabric) has a drawback that a connecting cable formed by attaching the tissue substitute artificial fiber cloth (fabric) to the cable body cannot be manufactured with high productivity and with high quality.

The present invention has been made in view of the above-mentioned circumstances, and it is an object of the present invention to provide a method of attaching a tissue substitute artificial fiber cloth where a connecting cable formed by attaching a tissue substitute artificial fiber cloth (fabric) to a cable body can be manufactured with high productivity and with high quality. It is another object of the present invention to provide a connecting cable for an auxiliary artificial heart having high productivity and high quality. It is still another object of the present invention to provide an auxiliary artificial heart system having excellent productivity as a whole system and having high reliability by adopting the connecting cable for an auxiliary artificial heart according to the present invention.

### Solution to Problem

[1] A method of attaching a tissue substitute artificial fiber cloth according to the present invention is a method of attaching a tissue substitute artificial fiber cloth where a connecting cable for an auxiliary artificial heart is disposed between a blood pump embedded in a body and a controller having a function of controlling the blood pump outside the body, and the tissue substitute artificial fiber cloth is attached to a surface of a cable body of the connecting cable so as to cover the surface of the cable body of the connecting cable at least between an inlet portion of the body and the blood pump between the controller and the blood pump, wherein the method includes:

a wrapping step of wrapping the tissue substitute artificial fiber cloth around the surface of the cable body such that a longitudinal direction of the tissue substitute artificial fiber cloth extends along a longitudinal direction of the cable body, wherein a long side of the tissue substitute artificial fiber cloth has at least a length between the inlet portion of the body and the blood pump and a short side of the tissue substitute artificial fiber cloth has a length longer than a circumferential length of the cable body,

an overlapping step of overlapping respective left and right marginal portions formed on a side where the respective left and right long sides are disposed by wrapping the tissue substitute artificial fiber cloth around the surface of the cable body; and

a welding step of welding root portions of the respective left and right marginal portions on a cable body side along the longitudinal direction of the cable body.

According to the method of attaching a tissue substitute artificial fiber cloth of the present invention, the tissue substitute artificial fiber cloth is attached to the cable body by the above-mentioned respective steps. Accordingly, compared to bonding by the conventional seaming, a time necessary for attaching the tissue substitute artificial fiber cloth can be considerably shortened. Further, according to the method of attaching a tissue substitute artificial fiber cloth of the present invention, the bonding portion after the welding step is finished has neither folded back portions nor a seam. Accordingly, a state is brought about where the tissue substitute artificial fiber cloth covers the surface of the cable body with a substantially uniform thickness and hence, it is also possible to acquire an advantageous effect that a high quality connecting cable can be obtained.

In this manner, according to the method of attaching a tissue substitute artificial fiber cloth of the present invention, it is possible to manufacture the connecting cable formed by attaching the tissue substitute artificial fiber cloth (fabric) to the cable body with high productivity and with high quality.

[2] In the method of attaching a tissue substitute artificial fiber cloth according to the present invention, it is preferable that the method further include, as a step preceding the wrapping step of wrapping the tissue substitute artificial fiber cloth, an adhesive sheet laminating step of laminating an adhesive sheet having both surfaces to which an adhesive agent is applied by coating to the surface of the cable body.

In this manner, the tissue substitute artificial fiber cloth is adhered to the cable body by way of the adhesive sheet having both surfaces to which an adhesive agent is applied by coating. Accordingly, it is possible to prevent the tissue substitute artificial fiber cloth from being displaced from the cable body and hence, the highly reliable connecting cable can be manufactured. With the use of the adhesive sheet, compared to an adhesive agent in a liquid form, the mesh of a fiber layer which forms the tissue substitute artificial fiber cloth is minimally clogged, and tissues of a person such as skin and flesh easily infiltrate into the inside of the fiber layer. Accordingly, it is possible to acquire an advantageous effect that the adhesion between the fabric and tissues of a person such as skin and flesh smoothly progresses.

[3] In the method of attaching a tissue substitute artificial fiber cloth according to the present invention, it is preferable that the method further include, as a step preceding the wrapping step of wrapping the tissue substitute artificial fiber cloth, an adhesive agent applying step of applying by coating an adhesive agent in a liquid form to the surface of the cable body.

In this manner, the tissue substitute artificial fiber cloth is adhered to the cable body by way of the adhesive agent in a liquid form. Accordingly, it is possible to prevent the tissue substitute artificial fiber cloth from being displaced from the cable body and hence, the highly reliable connecting cable can be manufactured. In this case, it is sufficient to only apply the adhesive agent in a liquid form to the cable body, it is possible to acquire an advantageous effect that the operation can be performed easily. It must be noticed, however, there is a type of adhesive agent in a liquid form which is liable to cause clogging of the mesh of the fiber layer which forms the tissue substitute artificial fiber cloth. Accordingly, it is preferable to use, as the adhesive agent in a liquid form, an adhesive agent in a liquid form which has a property of minimally causing clogging of the mesh of the fiber layer.

[4] In the method of attaching a tissue substitute artificial fiber cloth according to the present invention, it is preferable that the welding step be performed using an ultrasonic welder.

In this manner, by performing welding using the ultrasonic welder, it is possible to concentrically impart heat to a portion to be welded and hence, a periphery around the portion to be welded is minimally thermally affected. Accordingly, it is possible to prevent fibers of the tissue substitute artificial fiber cloth from being adversely thermally affected. Although various methods including heating tool welding, laser welding, high frequency welding, vibration welding and the like are considered as the welding method, in the case of welding the tissue substitute artificial fiber cloth, welding by ultrasonic welder has an advantageous effect that stringing after welding can be prevented.

[5] In the method of attaching a tissue substitute artificial fiber cloth according to the present invention, it is preferable that, in the welding step performed using the ultrasonic welder, ultrasonic vibration energy be imparted to the root portions of the respective left and right marginal portions on the cable body side in a state where the root portions are clamped and pressed by a horn and a base of the ultrasonic welder.

With such a step, it is possible to perform welding in the vicinity of the root portions of the marginal portions on the cable body side and hence, after welding is performed, it is possible to bring the tissue substitute artificial fiber cloth into close contact with the cable body.

[6] In the method of attaching a tissue substitute artificial fiber cloth according to the present invention, the welding step includes a step of cutting the marginal portions in addition to the welding.

Such a step can be realized by using the ultrasonic welder as the welding method. That is, cutting can be performed simultaneously with welding by optimizing the welding condition of welding using the ultrasonic welder. In this manner, by enabling cutting simultaneously with welding, after welding is performed, it is possible to eliminate a step of cutting the marginal portions by an operator or the like.

[7] A connecting cable for an auxiliary artificial heart according to the present invention is a connecting cable for an auxiliary artificial heart disposed between a blood pump embedded in a body and a controller having a function of controlling the blood pump outside the body, wherein the connecting cable for an auxiliary artificial heart includes:

a cable body in which a control signal line for controlling the blood pump is disposed; and a tissue substitute artificial fiber cloth which is attached to the cable body so as to cover a surface of the cable body at least between an inlet portion of the body and the blood pump between the controller and the blood pump, wherein

respective edge portions of the tissue substitute artificial fiber cloth on both left and right sides along a longitudinal direction of the tissue substitute artificial fiber cloth are welded to each other along a longitudinal direction of the connecting cable.

In the connecting cable for an auxiliary artificial heart of the present invention, the respective edge portions of the tissue substitute artificial fiber cloth attached to the cable body along a longitudinal direction of the tissue substitute artificial fiber cloth are bonded to each other by welding. Accordingly, the connecting cable for an auxiliary artificial heart of the present invention can exhibit high productivity compared to the conventional connecting cable where the respective edge portions of the tissue substitute artificial fiber cloth along the longitudinal direction are bonded to each other by seaming in a folded back state. Further, neither the folded back portions nor the seam exists at the bonding portion. Accordingly, the tissue substitute artificial fiber cloth attached to the cable body does not form local protrusions on a surface of the cable body and has an approximately uniform thickness in the circumferential direction of the cable body and hence, it is possible to provide the connecting cable with high quality.

In the connecting cable for an auxiliary artificial heart according to the present invention, it is preferable that the tissue substitute artificial fiber cloth be adhered to the cable body by way of an adhesive sheet having both surfaces to which an adhesive agent is applied by coating or by way of an adhesive agent in a liquid form. Further, it is preferable that welding of the respective edge portions of the tissue substitute artificial fiber cloth along the longitudinal direction be performed by an ultrasonic welder.

[8] An auxiliary artificial heart system according to the present invention is an auxiliary artificial heart system which includes:

a blood pump embedded in a body;

a controller having a function of controlling the blood pump outside the body; and

a connecting cable for an auxiliary artificial heart disposed between the blood pump and the controller, wherein

the connecting cable is the connecting cable for an auxiliary artificial heart described in the [7].

The auxiliary artificial heart system according to the present invention includes the connecting cable for an auxiliary artificial heart described in the above-mentioned [7] as the connecting cable. Accordingly, the auxiliary artificial heart system exhibits excellent productivity as the whole system and has high reliability.

### BRIEF DESCRIPTION OF DRAWINGS

FIG. 1 is a view for describing an auxiliary artificial heart system 100 according to an embodiment 1.

FIG. 2A to FIG. 2F are views for describing a method of attaching a fabric 220 according to the embodiment 1.

FIG. 3 is a perspective view showing a portion of a connecting cable 200 according to the embodiment 1 in an enlarged manner.

FIG. 4A and FIG. 4B are views for describing a method of attaching a fabric 220 according to an embodiment 2.

FIG. 5 is a view for describing an auxiliary artificial heart system 900 described in patent literature 1.

FIG. 6 is a view showing the auxiliary artificial heart system 900 which uses a connecting cable 950 formed by attaching a fabric 952 to a cable body 951.

FIG. 7A and FIG. 7B are views showing a main part of the connecting cable 950 shown in FIG. 6 in an enlarged manner.

### DESCRIPTION OF EMBODIMENTS

Embodiments of the present invention are described hereinafter.

### Embodiment 1

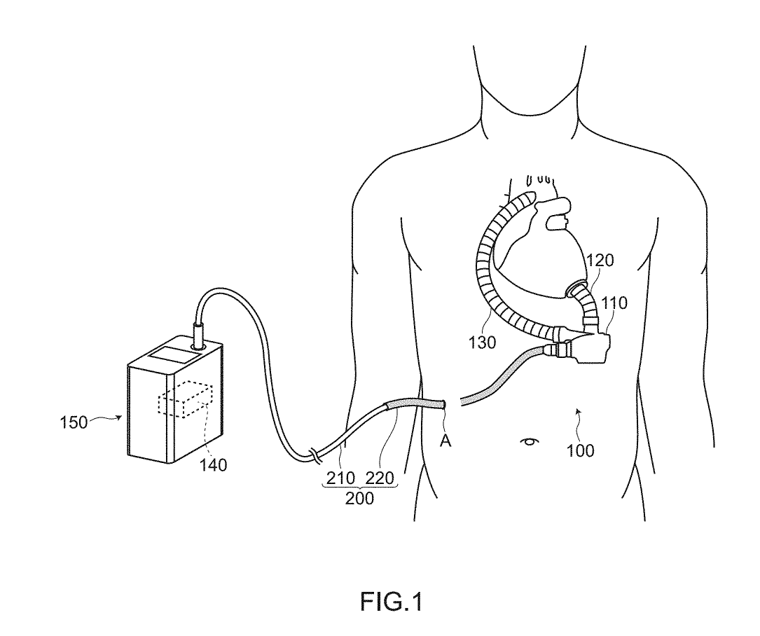

FIG. 1 is a view for describing an auxiliary artificial heart system 100 according to the embodiment 1. As shown in FIG. 1, the auxiliary artificial heart system 100 according to the embodiment 1 includes: a blood pump 110 embedded in a body of a patient; artificial blood vessels 120, 130 provided for connecting the blood pump 110 and a blood flow of a heart; a portable controller accommodating part 150 which accommodates a controller 140 which controls the blood pump 110 outside the body; and a connecting cable 200 for an auxiliary artificial heart disposed between the blood pump 110 and the controller 140 (connecting cable 200 for the auxiliary artificial heart according to the embodiment 1).

The connecting cable 200 for an auxiliary artificial heart according to the embodiment 1 (hereinafter also abbreviated as the connecting cable 200 according to the embodiment 1) includes: a cable body 210; and a fabric (tissue substitute artificial fiber cloth) 220 attached to the cable body 210 so as to cover a surface of the cable body 210.

The fabric 220 is attached to the cable body 210 so as to cover the surface of the cable body 210 at least between the inlet portion A of the body and the blood pump 110 between the blood pump 110 and the controller 140. When an existing fabric (assuming as a fabric having a long side length of 300 mm) is directly used in its original size, as described previously, in the case of a person having an ordinary physical build, the fabric 220 is brought into a state where the fabric 220 extends from the blood pump 110, passes through an inlet portion A of the body, and is exposed outside the body. A length of a portion of the fabric 220 exposed outside the body is approximately 100 mm in general although a difference exists among individuals to some extent. A method of attaching such a fabric 220 is described later.

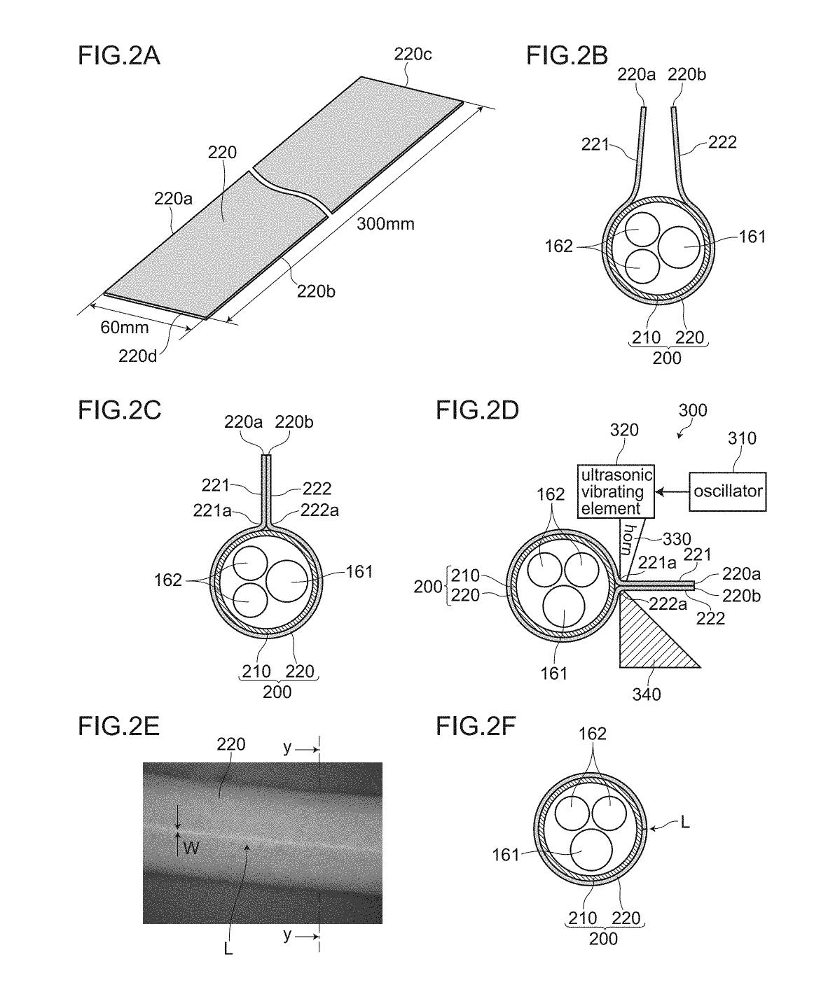

In the auxiliary artificial heart system 100 according to the embodiment 1, a purge liquid is circulated. The purge liquid has functions such as lubrication of the inside of the blood pump 110, cooling of the inside of the blood pump 110, and maintaining of the sealing performance of the inside of the blood pump 110. To this end, the auxiliary artificial heart system 100 also includes a purge liquid circulating device (not shown in the drawing) for circulating the purge liquid in the inside of the controller accommodating part 150. Further, in the inside of the cable body 210 of the connecting cable 200, besides an electric signal line 161 (see FIG. 2A to FIG. 2F) for controlling the blood pump 110, purge liquid circulating tubes 162 (for example, see FIG. 2B) for circulating the purge liquid are disposed. An outer diameter of the cable body 210 is approximately 10 mm.

FIG. 2A to FIG. 2F are views for describing a method of attaching the fabric 220 according to the embodiment 1. FIG. 2A to FIG. 2F show the respective steps. Hereinafter, the respective steps are specifically described. In FIG. 2A to FIG. 2F, the cable body 210, the fabric 220 and the like are not drawn by accurately reducing an actual size, but some parts are drawn in an exaggerating manner or in a contracted manner.

FIG. 2A is a view showing the fabric 220 used in the embodiment 1. The fabric 220 used in the embodiment 1 is an existing product formed of a non-woven fabric made of polyester or the like. Sizes of the fabric 220 are set such that long sides (sides in the longitudinal direction) 220a, 220b have a length of 300 mm, short sides (sides in the width direction) 220c, 220d have a length of 60 mm, and an average thickness is approximately 0.5 mm. Assuming that an outer diameter of the cable body 210 is 10 mm, the length of the short side 220c, 220d of the fabric 220 becomes larger than a length of a circumferential length (approximately 31.4 mm) of the cable body 210.

Next, a wrapping step is performed where such a fabric 220 is wrapped around the surface of the cable body 210 (see FIG. 2B). That is, the fabric 220 is wrapped around the surface of the cable body 210 such that the longitudinal direction of the fabric 220 (the direction along the long sides 220a, 220b) follows the longitudinal direction of the cable body 210. When the fabric 220 is wrapped around the surface of the cable body 210, respective left and right marginal portions 221, 222 are formed on left and right long sides of the fabric 220. With respect to the respective left and right marginal portions 221, 222, assuming that the circumferential length of the cable body 210 is approximately 32 mm and the length of the short side of the fabric 220 is 60 mm, the widths of the respective left and right marginal portions 221, 222 become approximately 10 several mm respectively.

Subsequently, an overlapping step is performed where the respective left and right marginal portions 221, 222 are made to overlap with each other (see FIG. 2C). Then, a welding step is performed where root portions 221a, 222a of the respective left and right marginal portions 221, 222 on a cable body 210 side are welded to each other along the longitudinal direction of the cable body 210 (see FIG. 2D).

The welding step is performed by using an ultrasonic welder 300. As shown in FIG. 2D, the ultrasonic welder 300 has, as main constitutional elements thereof, an oscillator 310, an ultrasonic vibrating element 320, a horn 330, a base 340 and the like. In FIG. 2D, the ultrasonic welder 300 is schematically shown.

In such an ultrasonic welder 300, ultrasonic vibration energy is imparted to the root portions 221a, 222a of the respective left and right marginal portions 221, 222 on the cable body 210 side in a state where the root portions 221a, 222a are clamped and pressed by the horn 330 and the base 340. By applying ultrasonic vibration energy in this manner, welding can be performed in the vicinity of the root portions 221a, 222a of the marginal portions 221, 222 on the cable body 210 side. Accordingly, it is possible to bring the fabric 220 into close contact with the cable body 210 after welding. In the method of attaching a fabric according to the embodiment 1, the ultrasonic welder 300 imparts ultrasonic vibration energy of approximately 15 KHz to the fabric 220. The horn 330 can perform welding to the fabric 220 at a length of approximately 50 mm per single welding operation.

In the method of attaching the fabric 220 according to the embodiment 1, the welding step is performed sequentially for respective welding units using approximately several tens of millimeters (50 mm, for example) as 1 welding unit from one end portion side (short side 220c side, for example) along the longitudinal direction of the fabric 220. By performing welding by optimizing welding conditions such as a load and a time at the time of imparting ultrasonic vibration energy, cutting can be performed simultaneously with welding. In this manner, the welding step shown in FIG. 2D also includes the step of cutting the marginal portions 221, 222 in addition to welding. Accordingly, the welding step shown in FIG. 2D may be also referred to as “welding and cutting step” hereinafter.

With the use of such an ultrasonic welder 300, the welding and cutting step is sequentially applied to the root portions 221a, 222a on the cable body 210 side for each 50 mm (1 welding unit) from one end portion side of the fabric. In performing the welding and cutting step for respective welding units, it is preferable to perform the welding and cutting step in a state where a more or less pulling force is applied to the respective left and right marginal portions 221, 222 of the fabric 220 toward the outside from a surface of the cable body 210. By performing the welding and cutting step in such a manner, the fabric 220 wrapped around the surface of the cable body 210 is released from a pulling force. Accordingly, after the welding and cutting step is finished, the bonding portion is brought into close contact with the surface of the cable body 210 and hence, the protrusion of the bonding portion can be suppressed.

Such a welding and cutting step is performed up to the other end portion (short side 220d side) of the fabric 220. FIG. 2E is a photograph of a surface of a portion of the fabric 220 after the welding and cutting step is finished for each welding unit in an enlarged manner. As shown in FIG. 2E, a white line L extending in the longitudinal direction on the surface of the fabric 220 is a bonding portion formed by welding and cutting in the welding and cutting step. A width W (referred to as welding width W) of the bonding portion (referred to as bonding portion L) is approximately 0.35 mm.

FIG. 2F is a cross-sectional view as viewed in an arrow direction y-y in FIG. 2E. As shown in FIG. 2F, after the welding and cutting step is finished, the surface of the fabric 220 minimally projects thus bringing about a state where the fabric 220 is covered on the surface of the cable body 210 with a substantially uniform thickness.

A time necessary for performing the above-mentioned welding and cutting step (a time for imparting ultrasonic vibration energy) for each welding unit is approximately 1.5 seconds. Accordingly, assuming that a length of 1 welding unit is 50 mm and a length of the fabric 220 in the longitudinal direction is 300 mm, a time necessary for the whole welding and cutting step (time for imparting ultrasonic vibration energy) in the longitudinal direction of the fabric 220 is approximately seconds in total. Accordingly, even when a time for preparation necessary for an operation in each welding unit takes 1 to 2 minutes, the welding and cutting step of the whole fabric 220 can be finished within 10 and several minutes.

As has been described heretofore, according to the method of attaching a fabric of the embodiment 1, the fabric 220 is attached to the cable body 210 through the above-mentioned respective steps. Accordingly, compared to bonding by conventional seaming, it is possible to considerably shorten a time necessary for attaching the fabric 220. Further, according to the method of attaching a fabric of the embodiment 1, after the welding and cutting step is finished, neither the folded back portions nor a seam exits in the bonding portion L. Accordingly, a state is brought about where the surface of the cable body 210 is covered with the tissue substitute artificial fiber cloth with a substantially uniform thickness and hence, it is also possible to obtain an advantageous effect that a high quality connecting cable can be obtained.

In this manner, according to the method of attaching a fabric of the embodiment 1, the connecting cable formed by attaching the fabric 220 to the cable body 210 can be manufactured as a connecting cable having high productivity and high quality.

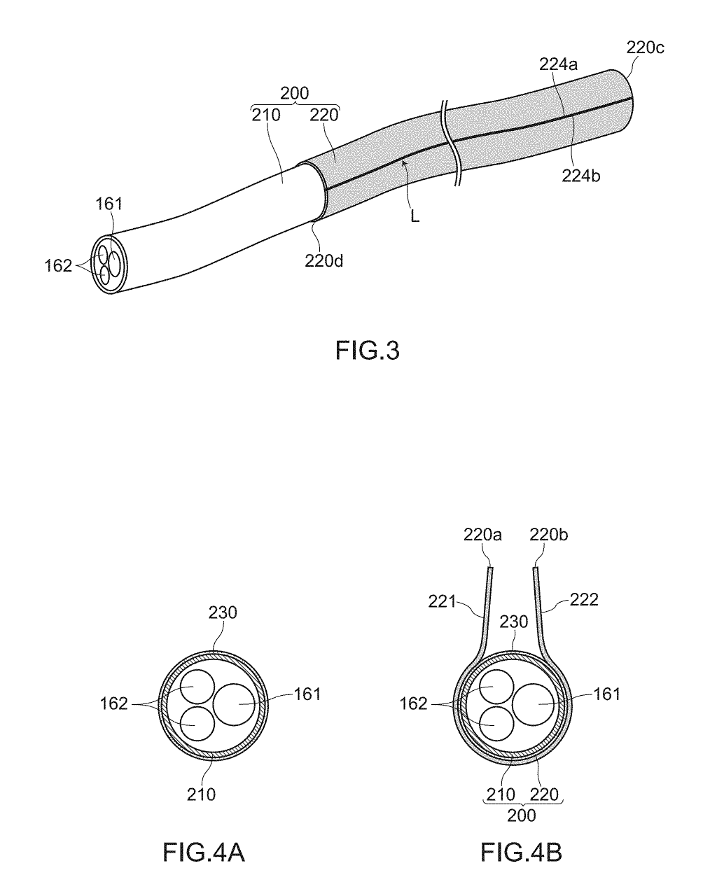

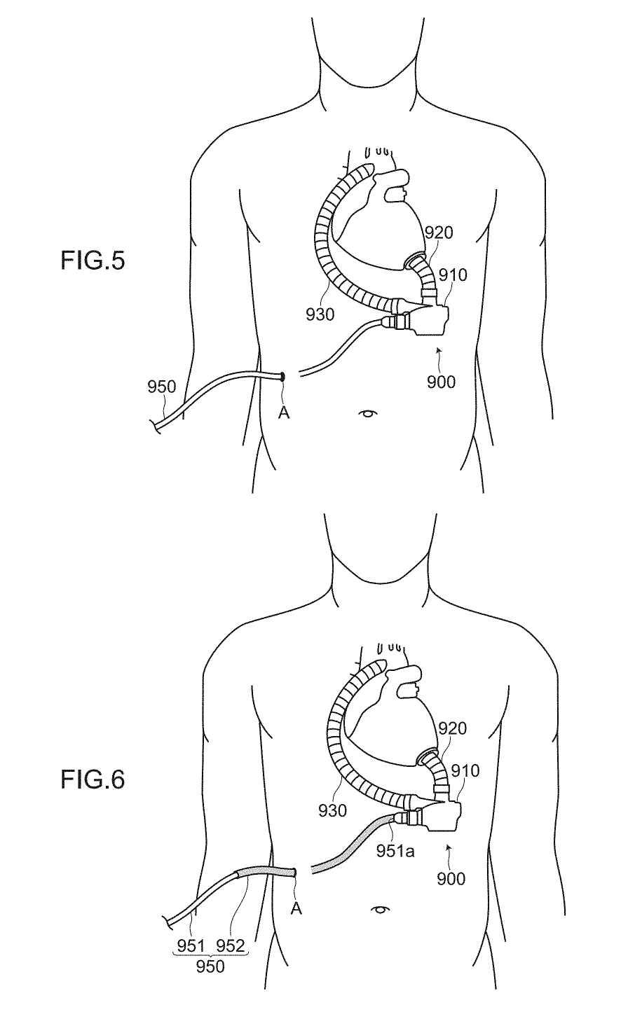

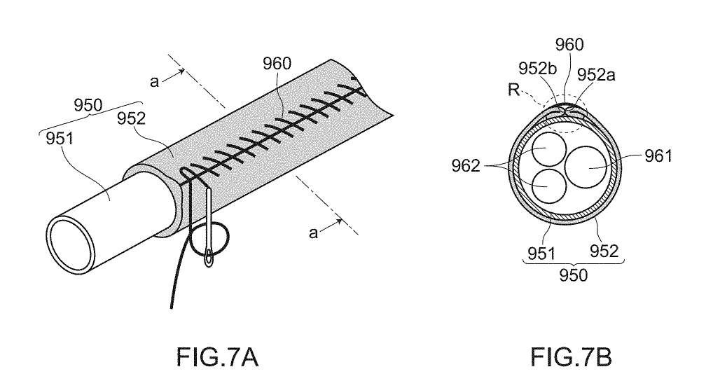

FIG. 3 is a perspective view showing a part of the connecting cable 200 according to the embodiment 1 in an enlarged manner. As shown in FIG. 3, the connecting cable 200 according to the embodiment 1 is configured such that the fabric 220 is attached to the cable body 210 so as to cover the surface of the cable body 210. The fabric 220 is configured such that edge portions 224a, 224b of the fabric 220 on both left and right sides along the longitudinal direction of the cable body 210 are bonded to each other by welding along the longitudinal direction of the fabric 220. Although the bonding portion L is indicated by a black bold line in FIG. 3, in the actual connecting cable 200, the bonding portion L is not so apparent as shown in FIG. 2E.

In this manner, in the connecting cable 200 for an auxiliary heart according to the embodiment 1, the fabric 220 is formed by bonding the edge portions 224a, 224b by welding. Accordingly, compared to the conventional connecting cable where bonding is performed by seaming, the connecting cable 200 has high productivity. Further, neither the folded back portions nor a seam exists at the bonding portion L and hence, the connecting cable 200 has high quality.

Further, the auxiliary artificial heart system 100 according to the embodiment 1 (see FIG. 1) includes, as the connecting cable, the connecting cable 200 according to the embodiment 1. Accordingly, it is possible to provide the auxiliary artificial heart system which exhibits excellent productivity at a whole, and has high reliability.

In the embodiment 1, the case is exemplified where the fabric 220 is directly attached to the surface of the cable body 210, and the description of a unit for fixing the fabric 220 to the cable body 210 is omitted. However, the connecting cable 200 may be provided with a fixing member for preventing the displacement of the fabric 220 relative to the cable body 210.

For example, in FIG. 3, on both one end portion (short side 220c) side and the other end portion (short side 220d) side of the fabric 220, the fabric 220 may be fixed to the cable body 210 respectively using a fixing member not shown in the drawing such as a clip or a band made of an elastic material. Further, besides the fixing members provided to one end portion (short side 220c) side and the other end portion (short side 220d) side, the fixing member may be provided at one portion or a plurality of portions between one end portion (short side 220c) and the other end portion (short side 220d). With such a configuration, the displacement of the fabric 220 relative to the cable body 210 can be prevented.

### Embodiment 2

In the above-mentioned embodiment 1, in attaching the fabric 220 to the cable body 210, the fabric 220 is directly attached to the surface of the cable body 210. However, in the embodiment 2, a fabric 220 is attached to a cable body 210 by way of an adhesive sheet.

Respective steps of a method of attaching a tissue substitute artificial fiber cloth according to the embodiment 2 are basically substantially equal to the corresponding steps of the method of attaching a tissue substitute artificial fiber cloth according to the embodiment 1 shown in FIG. 2A to FIG. 2F. However, in the method of attaching a tissue substitute artificial fiber cloth according to the embodiment 2, as a step preceding the wrapping step of wrapping the fabric 220 around the surface of the cable body 210 shown in FIG. 2B, an adhesive sheet laminating step of laminating an adhesive sheet 230 (see FIG. 4A and FIG. 4B) to the surface of the cable body 210 is performed.

The adhesive sheet 230 is a double-sided adhesive sheet where an adhesive agent is applied by coating to both surfaces thereof. Such an adhesive sheet 230 may be laminated to a whole region of the surface of the cable body 210 to which the fabric 220 is attached, or may be laminated to only a specific region within a region of the surface of the cable body 210 to which the fabric 220 is attached. With respect to the method of attaching a tissue substitute artificial fiber cloth according to the embodiment 2, the case is described where the adhesive sheet 230 is laminated to the whole region to which the fabric 220 is attached.

FIG. 4A and FIG. 4B are views for describing the method of attaching a tissue substitute artificial fiber cloth according to the embodiment 2. In FIG. 4A and FIG. 4B, FIG. 4A shows an adhesive sheet laminating step of laminating the adhesive sheet 230, and FIG. 4B shows a step corresponding to the wrapping step in FIG. 2B (the wrapping step of wrapping the fabric 220 around the surface of the cable body 210).

In this manner, the method of attaching a tissue substitute artificial fiber cloth according to the embodiment 2 differs from the method of attaching a tissue substitute artificial fiber cloth according to the embodiment 1 with respect to a point that, as a step preceding the wrapping step of wrapping the fabric 220 around the surface of the cable body 210 as shown in FIG. 2B, the adhesive sheet laminating step of laminating the adhesive sheet 230 (see FIG. 4A and FIG. 4B) to the surface of the cable body 210 is performed. The method of attaching a tissue substitute artificial fiber cloth according to the embodiment 2 is substantially equal to the method of attaching a tissue substitute artificial fiber cloth according to the embodiment 1 with respect to other steps shown in FIG. 2A to FIG. 2F. Accordingly, in this embodiment 2, the adhesive sheet laminating step is mainly described, and the explanation of other steps is abbreviated or omitted. Hereinafter, the method of attaching a tissue substitute artificial fiber cloth according to the embodiment 2 is described with reference to FIG. 2A to FIG. 2F and FIG. 4A and FIG. 4B.

First, the fabric 220 is prepared (see FIG. 2A) and, thereafter, the adhesive sheet 230 is laminated to a predetermined region of the surface of the cable body 210 (the whole region to which the fabric 220 is attached) (see FIG. 4A). It is preferable that a thickness of the adhesive sheet 230 be set to a thickness such that, when the adhesive sheet 230 is laminated to the cable body 210, the thickness of the adhesive sheet 230 minimally affects an outer diameter of the connecting cable 200 including the fabric 220. For example, the thickness of the adhesive sheet 230 may be set to a value which falls within a range of from approximately 5 μm to 400 μm. However, it is preferable to set the thickness of the adhesive sheet 230 to a value which falls within a range of from approximately 100 μm to 200 μm from a viewpoint of operability and from a viewpoint of a fixing force of the fabric 220 to the cable body 210.

Subsequently, the wrapping step of wrapping the fabric 220 around a surface side of the adhesive sheet 230 which is laminated to the cable body 210 is performed (see FIG. 4B)). Then, an overlapping step of overlapping respective left and right marginal portions 221, 222 is performed (see FIG. 2C) and, thereafter, a welding and cutting step is performed (see FIG. 2D).

According to the method of attaching a tissue substitute artificial fiber cloth of the embodiment 2, the fabric 220 is adhered to the surface of the cable body 210 by way of the adhesive sheet 230 and hence, it is possible to prevent the displacement of the fabric 220 relative to the cable body 210. Accordingly, the method of attaching a tissue substitute artificial fiber cloth according to the embodiment 2 can acquire an advantageous effect that a highly reliable connecting cable can be obtained in addition to the advantageous effects acquired by the method of attaching a tissue substitute artificial fiber cloth according to the embodiment 2.

Further, the connecting cable for an auxiliary artificial heart (referred to as a connecting cable 200′) according to the embodiment 2 is configured such that the fabric 220 is adhered to the surface of the cable body 210 by way of the adhesive sheet 230. Accordingly, the connecting cable 200′ for an auxiliary artificial heart according to the embodiment 2 can acquire an advantageous effect that a more reliable connecting cable is obtained in addition to the advantageous effects obtained by the connecting cable 200 for an auxiliary artificial heart according to the embodiment 1. The connecting cable 200′ for an auxiliary artificial heart according to the embodiment 2 has substantially the same external appearance as the connecting cable 200 for an auxiliary artificial heart according to the embodiment 1 (see FIG. 1 and FIG. 3) and hence, the illustration of the connecting cable 200′ for an auxiliary artificial heart according to the embodiment 2 is omitted.

The auxiliary artificial heart system according to the embodiment 2 (referred to as auxiliary artificial heart system 100′) includes, as a connecting cable, the connecting cable 200′ for an auxiliary artificial heart according to the embodiment 2. Accordingly, the auxiliary artificial heart system 100′ according to the embodiment 2 can acquire an advantageous effect that the auxiliary artificial heart system 100′ becomes a more reliable auxiliary artificial heart system in addition to the advantageous effects acquired by the auxiliary artificial heart system 100 according to the embodiment 1. The auxiliary artificial heart system 100′ according to the embodiment 2 has substantially the same external appearance as the auxiliary artificial heart system 100 according to the embodiment 1 (see FIG. 1) and hence, the illustration of the auxiliary artificial heart system 100′ according to the embodiment 2 is omitted.

In the embodiment 2, the case is exemplified where the adhesive sheet 230 is used. However, an adhesive agent in a liquid form may be applied by coating to the cable body 210 in place of the adhesive sheet 230. In the case of the adhesive agent in a liquid form, it is sufficient to simply apply the adhesive agent in a liquid form by coating to the cable body 210 and hence, it is possible to acquire an advantageous effect that an operation becomes easy compared to lamination of the adhesive sheet 230 to the cable body 210. On the other hand, in the case of an adhesive agent in a liquid form, a control of a coating amount of the adhesive agent is difficult. Further, an adhesive agent in a liquid form may easily cause a capillary phenomenon depending on a kind of the adhesive agent, and when such an adhesive agent in a liquid form is used, the adhesive agent infiltrates into the inside of a fiber layer of the fabric 220 thus giving rise to a possibility that a function of the fabric 220 is adversely affected.

That is, when an adhesive agent in a liquid form enters the inside of the fiber layer of the fabric 220 and is solidified thereafter, the mesh of the fiber layer which form the fabric 220 is clogged. Accordingly, tissues of a person such as skin and flesh hardly infiltrate into the inside of the fiber layer thus giving rise to a possibility that the adhesion between the fabric 220 and tissues of a person such as skin and flesh becomes difficult. Such a drawback does not occur with respect to the adhesive sheet and hence, the adhesive sheet is advantageous in view of this point . However, even with respect to an adhesive agent in a liquid form, provided that an adhesive agent in a liquid form having a property of minimally causing a capillary phenomenon (for example, high viscosity) is used, such a drawback can be obviated.

The present invention is not limited to the above-mentioned embodiments, and various modifications can be carried out without departing from the gist of the present invention. For example, the following modifications can be also carried out.

(1) In the above-mentioned respective embodiments, the existing product where the fabric has a length of 300 mm is used. Accordingly, as shown in FIG. 1, in the case of a person having an ordinary physical build, a part (approximately 100 mm) of the fabric is exposed to the outside of the body. However, by suitably selecting or setting the length of the fabric, it is also possible to set a length of the fabric exposed to the outside of the body to an optimal length.
(2) In the above-mentioned respective embodiments, in performing the welding and cutting step, welding is sequentially performed for respective welding units where a welding length of one time (1 welding unit) is set to 50 mm. However, by suitably setting a shape and a size of the horn of the ultrasonic welder and the base, it is possible to increase the welding unit. In this case, welding of the whole fabric can be performed more efficiently thus further enhancing productivity of the connecting cable for an auxiliary artificial heart.
(3) In the above-mentioned respective embodiments, the case is exemplified where the auxiliary artificial heart system 100 is configured such that a purge liquid is circulated in the inside of the blood pump 110. However, the present invention is also applicable to a case where a fabric is attached to a connecting cable used in an auxiliary artificial heart system where a purge liquid is not circulated.
(4) In the above-mentioned embodiment 2, the case is exemplified where the adhesive sheet 230 is laminated to the whole region corresponding to the region to which the fabric 220 is attached. However, the present invention is not limited to such a case and, as described previously, the adhesive sheet 230 may be laminated to the specific region in the region to which the fabric 220 is attached on the surface of the cable body 210. For example, the adhesive sheet may be laminated to a region in the vicinity of one end portion (short side 220c) of the fabric 220 in the longitudinal direction, a region in the vicinity of the other end portion (short side 220d) of the fabric 220 in the longitudinal direction, and regions at a plurality of positions between one end portion (short side 220c) and the other end portion (short side 220d). Such a configuration is also applicable to a case where an adhesive agent in a liquid form is used in place of the adhesive sheet 230 substantially in the same manner.

1. A method of attaching a tissue substitute artificial fiber cloth where a connecting cable for an auxiliary artificial heart is disposed between a blood pump embedded in a body and a controller having a function of controlling the blood pump outside the body, and the tissue substitute artificial fiber cloth is attached to a surface of a cable body of the connecting cable so as to cover the surface of the cable body of the connecting cable at least between an inlet portion of the body and the blood pump between the controller and the blood pump, the method comprising:
a wrapping step of wrapping the tissue substitute artificial fiber cloth around the surface of the cable body such that a longitudinal direction of the tissue substitute artificial fiber cloth extends along a longitudinal direction of the cable body, wherein a long side of the tissue substitute artificial fiber cloth has at least a length between the inlet portion of the body and the blood pump and a short side of the tissue substitute artificial fiber cloth has a length longer than a circumferential length of the cable body,
an overlapping step of overlapping respective left and right marginal portions formed on a side where the respective left and right long sides are disposed by wrapping the tissue substitute artificial fiber cloth around the surface of the cable body; and
a welding step of welding root portions of the respective left and right marginal portions on a cable body side along the longitudinal direction of the cable body.

2. The method of attaching a tissue substitute artificial fiber cloth according to claim 1, further comprising, as a step preceding the wrapping step of wrapping the tissue substitute artificial fiber cloth to the connecting cable for the auxiliary artificial heart, an adhesive sheet laminating step of laminating an adhesive sheet having both surfaces to which an adhesive agent is applied by coating to the surface of the cable body.

3. The method of attaching a tissue substitute artificial fiber cloth according to claim 1 further comprising, as a step preceding the wrapping step of wrapping the tissue substitute artificial fiber cloth, an adhesive agent applying step of applying by coating an adhesive agent in a liquid form to the surface of the cable body.

4. The method of attaching a tissue substitute artificial fiber cloth according to claim 1, wherein the welding step is performed using an ultrasonic welder.

5. The method of attaching a tissue substitute artificial fiber cloth according to claim 4, wherein in the welding step performed using the ultrasonic welder, ultrasonic vibration energy is imparted to the root portions of the respective left and right marginal portions on the cable body side in a state where the root portions are clamped and pressed by a horn and a base of the ultrasonic welder.

6. The method of attaching a tissue substitute artificial fiber cloth according to claim 5, wherein the welding step includes a step of cutting the marginal portions in addition to the welding.

7. A connecting cable for an auxiliary artificial heart disposed between a blood pump embedded in a body and a controller having a function of controlling the blood pump outside the body, the connecting cable for an auxiliary artificial heart comprising:
a cable body in which a control signal line for controlling the blood pump is disposed; and a tissue substitute artificial fiber cloth which is attached to the cable body so as to cover a surface of the cable body at least between an inlet portion of the body and the blood pump between the controller and the blood pump, wherein
respective edge portions of the tissue substitute artificial fiber cloth on both left and right sides along a longitudinal direction of the tissue substitute artificial fiber cloth are welded to each other along a longitudinal direction of the connecting cable.

8. An auxiliary artificial heart system comprising:
a blood pump embedded in a body;
a controller having a function of controlling the blood pump outside the body; and
a connecting cable for an auxiliary artificial heart disposed between the blood pump and the controller, wherein
the connecting cable is the connecting cable for an auxiliary artificial heart described in claim 7.
